(19) United States
(12) Patent Application Publication
(10) Pub. No.: US 2018/0206786 A1
(43) Pub. Date: Jul. 26, 2018

(54) SYSTEM AND METHOD FOR MEASURING EFFECTIVENESS OF AUTONOMIC NEUROSTIMULATION

(71) Applicant: BIOTRONIK SE & Co. KG, Berlin (DE)

(72) Inventors: Christopher S. de VOIR, Tigard, OR (US); Andrew B. KIBLER, Lake Oswego, OR (US); Dirk MUESSIG, West Linn, OR (US)

(73) Assignee: BIOTRONIK SE & Co. KG, Berlin (DE)

(21) Appl. No.: 15/879,075

(22) Filed: Jan. 24, 2018

Related U.S. Application Data
(60) Provisional application No. 62/449,603, filed on Jan. 24, 2017.

(51) Int. Cl. A61B 5/00; A61N 1/36; A61B 5/053; A61B 5/11; A61B 7/02

(52) U.S. Cl. CPC A61B 5/4848; A61N 1/36114; A61B 5/0538; A61B 2562/0219; A61B 7/023; A61B 5/6869; A61B 5/7239; A61B 5/1107

(57) ABSTRACT

A system for evaluating an efficacy of vagus nerve stimulation is provided, wherein the system has a neurostimulator that is configured to perform vagus nerve stimulation, and a measuring component for evaluating the efficacy based on at least one parameter that is indicative of a myocardial contractile state of the heart. A corresponding method is also provided.

This nonprovisional application claims priority to U.S. Provisional Application No. 62/449,603, which was filed on Jan. 24, 2017, and which is herein incorporated by reference.

### BACKGROUND OF THE INVENTION

### Field of the Invention

The present invention relates to a system and a method for evaluating efficacy of vagus nerve stimulation (VNS).

### Description of the Background Art

Vagus nerve stimulation (VNS) is being studied for a variety of therapeutic applications, many of which take advantage of its increase in parasympathetic tone on the heart. Vagus nerve stimulation (VNS) has been shown to improve outcomes in cardiac ischemia, tachy-arhythmias, inflammatory diseases, and heart failure. A clear and rapid measure of stimulation efficacy is desired which allows stimulation titration and parameter optimization. Such a measure would also allow for improved system battery life and decreased side effects as a result of optimized stimulation.

Usually, measuring the efficacy of VNS with regard to an increase in cardioactive parasympathetic tone requires imaging systems that perform echocardiography, angiography, or plethysmography. Moreover, it is normally required to apply high stimulation amplitudes in order to obtain a measurable effect.

Furthermore, long-term measures of stimulation efficacy in the treatment of heart failure including blood vessels of inflammatory cytokine marker Pro-BNP, NYHA (New York Heart Association) heart failure class, ventricular diameter changes, or self-reported measures such as MLWHF (Minnesota Living With Heart Failure) score.

Reported methods of rapid cervical level VNS feedback include laryngeal activation measures via electromyography (EMG) or external accelerometer. These methods operate by observing side effects caused by activation of the recurrent laryngeal fibers contained in the cervical level vagal trunk, and are not a direct measure of the cardioprotective effect of parasympathetic activation.

Existing solutions for obtaining rapid measures of cardiac VNS effect require significant external or invasive equipment (echocardiography, RV plethysmography, angiography), or undesirably high VNS levels (heart rate). Other known solutions (laryngeal activation measures) are nonspecific and currently impractical for continuous use.

Long-term heart failure status measures of efficacy do not allow rapid therapy optimization and can be subjective.

Furthermore, U.S. Pat. No. 8,939,904 B2, which is incorporated herein by reference, discloses a monitoring device for predicting cardiovascular anomalies, wherein the device may acquire a value change of a hemodynamic parameter, which occurs as a result of a detected value change of a state parameter.

### SUMMARY OF THE INVENTION

It is therefore an object of the present invention to provide a system and a method for assessing efficacy of VNS.

According to an exemplary embodiment, a system for evaluating an efficacy of vagus nerve stimulation is disclosed, wherein the system comprises a neurostimulator that is configured to perform vagus nerve stimulation, and a measuring component for evaluating the efficacy based on at least one parameter that is indicative of a myocardial contractile state (for example but not limited to inotropic and/or lusitropic states) of the (e.g. human) heart of the patient.

Particularly, both the neurostimulator and the measuring component are implantable into the body of the patient. Further, particularly, the measuring component is configured to communicate data to the neurostimulator. Particularly, the system may be configured to conduct bidirectional communication between the neurostimulator and the measuring component. The communication may be carried out via a line or wireless, for example based on radio frequency (RF), acoustic signals, optical signals, changes of a magnetic or electric field or the like. The data communicated between the neurostimulator and the measuring component may comprise measured values from the neurostimulator and the measuring component as well as information on stimulation parameters from the neurostimulator.

According to an embodiment of the system according to the present invention, the parameter can be, for example, one of intracardiac impedance (Z); ventricular wall motion; heart sounds, low frequency fluid motion acoustic signals; and/or a parameter derived therefrom, wherein the measuring component is configured to measure the parameter.

Furthermore, according to an embodiment of the system according to the present invention, the intracardiac impedance can be measured in an unipolar manner, and the measuring component can comprise an electrode having a tip that is configured to be arranged at a location in the heart, preferably the apex of the right ventricle (RV) of the heart for measuring the intracardiac impedance. According to an embodiment of the invention, the electrode tip may be arranged at any location in or around the heart, its chambers, tissue layers, vessels, or vicinity that would source a measure capable of evaluating VNS efficacy.

Particularly, the measuring component can be configured to measure the impedance in a unipolar manner, which involves measuring the impedance between the tip and a counter electrode, wherein the counter electrode may be provided by a housing of the measuring component.

Particularly, according to an embodiment of the system according to the present invention, the neurostimulator may form a unit that is separate from (and particularly in communication with) the measuring component (i.e. these two components comprise separate housings). However, the neurostimulator and the measuring component may also be integrated into a single housing. The communication may be carried out via a line or wireless, for example based on radio frequency (RF), acoustic signals, optical signals, changes of a magnetic or electric field or the like.

Particularly, the intracardiac impedance signal obtained with this configuration can be determined by conductivity changes in the vicinity of the electrode tip, wherein such conductivity changes occur due to changes in the percentage of myocardial tissue volume to blood volume in the surrounding of the electrode tip during isovolumetric contraction and ejection. Therefore, the impedance signal is indicative of the geometrical changes of the myocardium during contraction. Thus, the intracardiac impedance may serve as a parameter or may be used to derive a parameter that correlates well with contractility of the heart. Since the contraction pattern of the heart is altered under sympathetic influence, the intracardiac impedance contains information about the autonomic nervous system (ANS).

Furthermore according to an embodiment of the system according to the present invention the neurostimulator can be configured to perform vagus nerve stimulation (VNS) by activating parasympathetic ganglia in the heart, for example, either through stimulation of the vagus nerve or direct ganglia stimulation, particularly by means of electrical impulses generated by the neurostimulator and applied to the vagus nerve or ganglia by means of at least one or a plurality of stimulation electrodes of the neurostimulator.

Furthermore, according to an embodiment of the system according to the present invention, the system is configured to determine the parameter by means of the measuring component during diastole and/or systole of the cardiac cycle.

Particularly, in an embodiment, the system can be configured to repeatedly determine the parameter during vagus nerve stimulation performed by the system and in the absence of vagus nerve stimulation and to compare the parameter obtained during vagus nerve stimulation with the parameter obtained in the absence of vagal nerve stimulation for evaluating the efficacy of the vagus nerve stimulation (e.g. the response of the heart's lusitropy and inotropy to the stimulation). Particularly, the relaxation of the myocardium around the electrode tip corresponds to lusitropy, whereas the contraction of the myocardium around the tip corresponds to inotropy. The comparison may be performed by evaluating the parameter with respect to at least one of a reference value, an upper and lower limit, a statistical moment, one or more direct or derived value from the same sensor at another time in the heart cycle, a direct or derived value from another sensor or sensors, or the state of a therapy device.

In this way, the measurements performed by the system provide real-time or trended implant-based feedback of the change in cardiac dynamics due to neuromodulation.

The comparative change of “neurostimulation on” vs. “neurostimulation off” of these parameters/measures provides a rapid assessment of neurostimulation efficacy.

Particularly, the vagus nerve stimulation is delivered by the system with a duty cycle ‘on’ period (vagus nerve stimulation present) of 10-30 seconds and an ‘off’ period (vagus nerve stimulation absent) of 30 seconds to 5 minutes. The measurements of the intracardiac impedance can be taken during VNS ‘on’ periods and compared against VNS ‘off’ periods, allowing 5-60 seconds for the VNS effect to wash out. Particularly, the measured parameters will provide information on cardiac function as well as allow guided titration of vagus nerve stimulation after implant for optimal efficacy. Such titration may include not only stimulation parameters by also a selection of the stimulation electrodes in case of a multi-electrode system (see also above).

Furthermore, according to an embodiment of the system according to the present invention, the system comprises an accelerometer configured to detect movements of the patient, wherein particularly the system is configured to conduct an algorithm which generates a measure for the patient's activity. Advantageously, the patient's activity trend will allow for long-term efficacy evaluation via its approximation of quality of life through activity.

Furthermore, according to an embodiment of the system according to the present invention, the derived parameter measured/determined by the system can correspond to one: a time period over which the waveform of one of the intracardiac impedance (Z), ventricular wall motion, heart sounds or acoustic signals remains flat during the isovolumetric relaxation period, wherein particularly when lusitropy is improving due to the vagus nerve stimulation (VNS), the amount of time spent in this flat region will shorten in a pre-defined manner which is considered as a VNS having sufficient efficacy. The efficacy may be evaluated by any method suitable to evaluate a sample against a value direct or derived from a reference, limits, an expectation, or a device state. Appropriate scaling of such a result is dependent on the distribution of the data and clinical standards.—a time period between the closure of the aortic valve and the opening of the Mitral valve, which period of time is estimated by means of the first-order derivative of one of the measured intracardiac impedance (Z), ventricular wall motion, heart sounds or acoustic signals waveform, wherein particularly this time period between these two changes in a valve state is reduced with effective VNS, wherein when the period of time is reduced in a pre-defined manner, the efficacy is considered as being sufficient.

For example, an approach to evaluate waveform flatness is to determine amplitude variation of the waveform. For instance, a waveform can be declared as flat when the amplitude has not varied for more than a certain percentage within a predetermined time. An exemplary process for defining flatness of an impedance waveform is given in the following: The impedance signal is sampled at a certain rate. A linear model is generated for the time/impedance pairs (such as z(t)˜m*t+b) for t=0, 1, . . . , n). An analysis of variance is applied and the resulting parameters are tested against the hypothesis that they differ significantly from the null hypothesis of a horizontal line (that is ‘flat’). Since real physiologic signals even when sampling a ‘flat’ region will likely contain some non-zero offset and variance, these statistical moments can be tested with p-value and F-statistic to see if the ‘flat’ region varies significantly from an ideal horizontal line at a fixed direct current value.

Yet another aspect of the present invention relates to a method for evaluating an efficacy of vagus nerve stimulation, particularly using a system according to the invention, wherein an efficacy of vagus nerve stimulation is evaluated based on at least one parameter that is indicative of a myocardial contractile state of the heart.

Particularly, the vagus nerve stimulation itself is not a step or part of the claimed method, which is dedicated to measuring the effect of such a stimulation that has been applied beforehand.

For example, according to an embodiment of the method according to the present invention, the parameter can be: intracardiac impedance (Z), ventricular wall motion, heart sounds, low frequency fluid motion acoustic signals, and/or a parameter derived therefrom. Also, the parameter can be measured.

Furthermore, according to an embodiment of the method according to the present invention, the intracardiac impedance is unipolar intracardiac impedance that is measured using an electrode having a tip that has been arranged at the right ventricular apex. Particularly, arranging the tip at the apex can be conducted beforehand.

Further, according to an embodiment of the method according to the present invention, the unipolar impedance may be measured between the tip and a counter electrode, which counter electrode may be provided by a housing of a measuring component.

Furthermore, according to an embodiment of the method according to the present invention, the parameter can be determined during diastole and/or systole of the cardiac cycle.

Furthermore, according to an embodiment of the method according to the present invention, the parameter is repeatedly determined during vagus nerve stimulation and in the absence of vagus nerve stimulation, and wherein the parameter obtained during vagus nerve stimulation is compared to the parameter obtained in the absence of vagus nerve stimulation for evaluating the efficacy.

Furthermore, according to an embodiment of the method according to the present invention, a movement of the body of the patient is detected in addition, and an activity measure of the patient is derived from the detected movements (see also above).

Furthermore, according to an embodiment of the method according to the present invention, the derived parameter can correspond to: a time period over which one of the intracardiac impedance (Z), ventricular wall motion, heart sounds or acoustic signals remains flat during the isovolumetric relaxation period, wherein particularly when lusitropy is improving due to the vagus nerve stimulation (VNS), the amount of time spent in this flat region will shorten; and/or a time period between the closure of the aortic valve and the opening of the Mitral valve, which time period is estimated by means of the first-order derivative of one of the measured intracardiac impedance (Z), ventricular wall motion, heart sounds or acoustic signals, wherein the time period between these two changes in valve state is reduced with effective VNS.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a system/method according to the present invention;

FIG. 2 shows a heart suffering from compromised lusitropy due to increased sympathetic tone and the effect of VNS;

FIG. 3 shows traces of a typical electrocardiogram, the corresponding intracardiac impedance and its first derivative, wherein the systole is highlighted; and

FIG. 4 shows traces of a typical electrocardiogram, the corresponding intracardiac impedance and its first derivative, wherein the diastole is highlighted.

### DETAILED DESCRIPTION

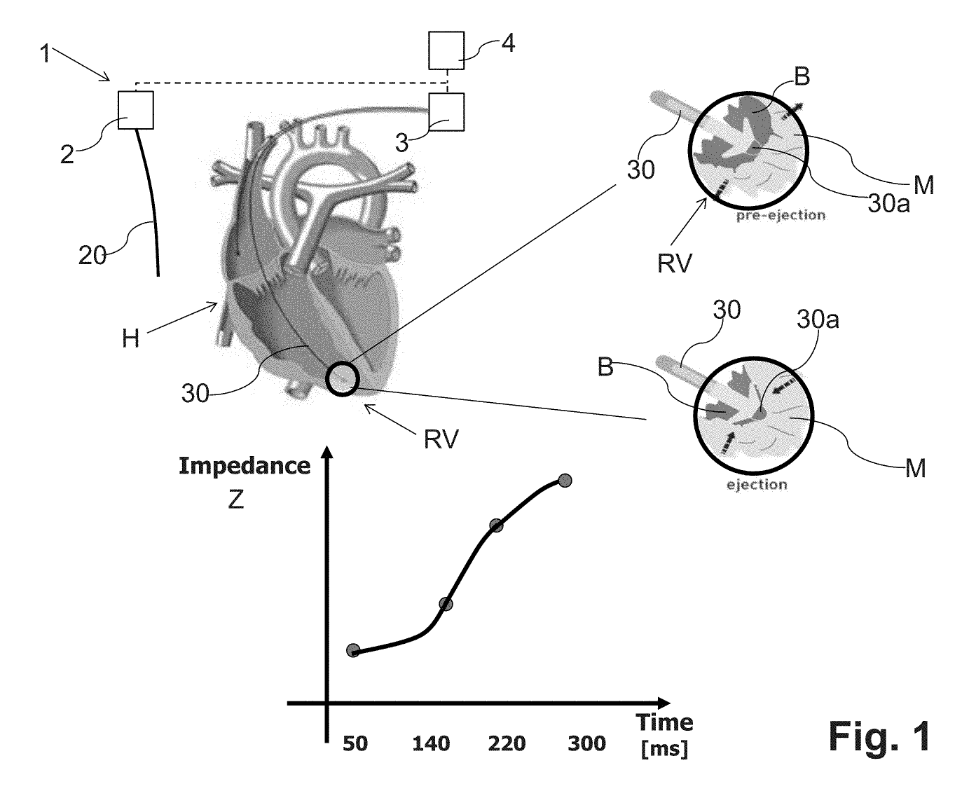

FIG. 1 shows a system 1 for evaluating an efficacy of vagus nerve stimulation VNS according to the present invention. The system 1 comprises a neurostimulator 2 that is configured to perform vagus nerve stimulation, and a measuring component 3 for evaluating the efficacy based on at least one parameter Z that is indicative of a myocardial contractile state of the heart H (e.g. as shown in the details of FIG. 1). According to an embodiment of the invention, measuring component 3 comprises or is connected to an electrode, wherein the electrode is configured to perform measurements of electrical parameters of a tissue. Measuring component 3 may also comprise processing components for computation and evaluation of the measured electrical parameters, as e.g. deriving parameter Z and evaluating the efficacy based on at least one parameter Z.

In addition, the proposed system 1 may incorporate an accelerometer 4 which measures patient activity and an algorithm which generates a patient activity trend. The patient activity trend will allow for long-term efficacy evaluation via its approximation of quality of life through activity.

Particularly, the parameter Z is an intracardiac impedance Z (or a parameter derived therefrom), wherein the measuring component 3 is configured to measure the intracardiac impedance Z, particularly by means of an electrode 30 in an unipolar configuration, wherein the electrode 30 comprises a tip 30a that is particularly arranged at the apex of the right ventricle RV. Using the unipolar electrode configuration for measuring impedance Z, the electrical path conducts through myocardium and blood, wherein the myocardium exhibits electrical impedance which is higher than blood. Consequently, the measured value of impedance Z depends on the relation of myocardium to blood within the electrical measurement path. That relation of myocardium to blood is depended on the cardiac contraction state, which is explained further in the following. FIG. 1 includes two detail illustrations which show the electrode 30 with electrode tip 30a in two different contraction states of the heart. In the ‘pre-ejection’ phase, the cardiac ventricles are filled with blood and the myocardium is relaxed. As a result, there is a comparatively high volume of blood B and low portion of myocardium in the vicinity of electrode tip 30a, resulting in a measured impedance Z which is low. In the ‘ejection’ phase of the heart, the blood is pumped out of the ventricles and the myocardium is in a contracted state. Consequently, a comparatively high portion of myocardium and small portion of blood surrounds electrode tip 30a, resulting in a measured impedance Z which is high.

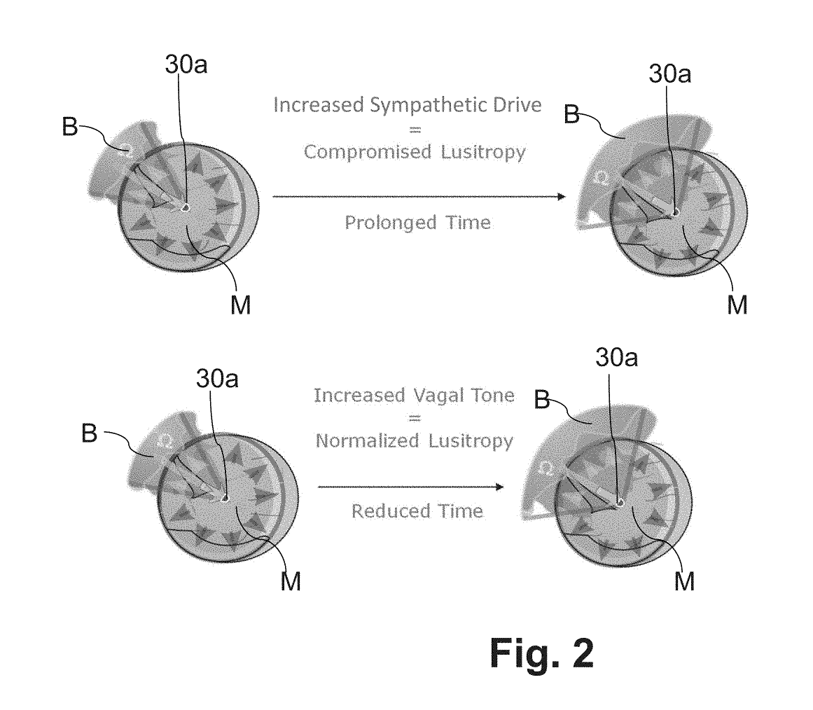

The ability of the myocardium to change from the contracted state to the relaxed state is called cardiac lusitropy. When a patient suffers from disturbed, i.e. increased sympathetic drive, cardiac lusitropy is compromised in a way which is illustrated in FIG. 2. The four images in FIG. 2 each show an intra-cardiac lead tip 30a embedded in the RV apex at the peak of contraction. The myocardium M contracts around the lead tip 30a, enveloping it in cardiac tissue which exhibits higher measurable electrical impedance than blood B. The upper half of FIG. 2 depicts schematically cardiac contraction behavior influenced by an increased sympathetic drive and how this is represented in the impedance measurements: Cardiac lusitropy is compromised in a way that the transition time for a change of the cardiac contraction state from high impedance (upper left image) to low impedance (upper right image) is prolonged, resulting in a prolonged time for the ventricle to relax and allowing passive phase diastolic pre-filling of the ventricle. In the lower half of FIG. 2, the effect of VNS therapy on a patient suffering from increased sympathetic tone is shown: Due to an increased vagal tone, the transition time from high impedance (lower left image) to low impedance (lower right image) is reduced, resulting in normalized cardiac lusitropy.

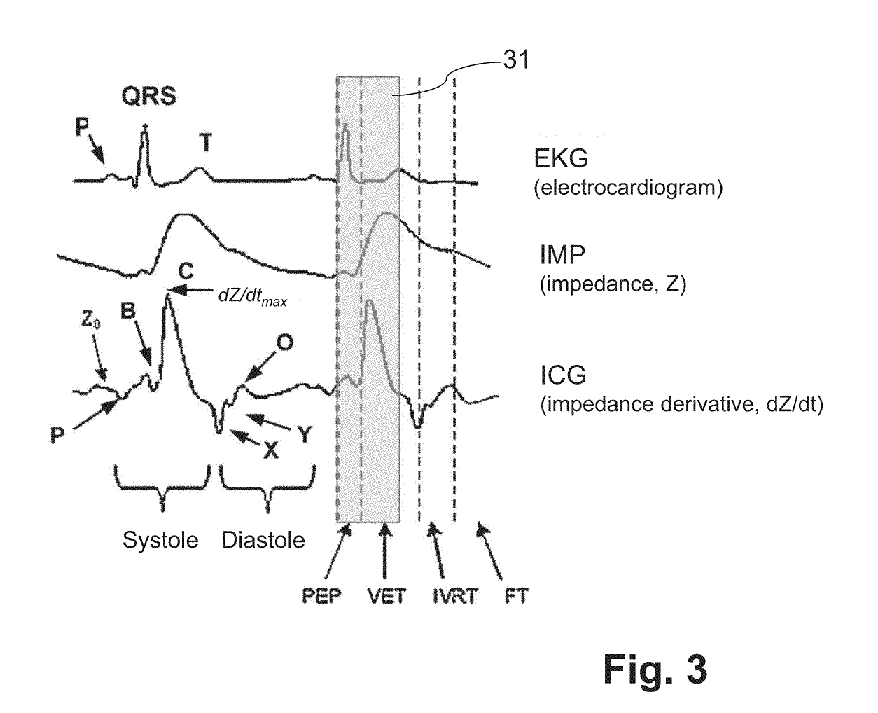

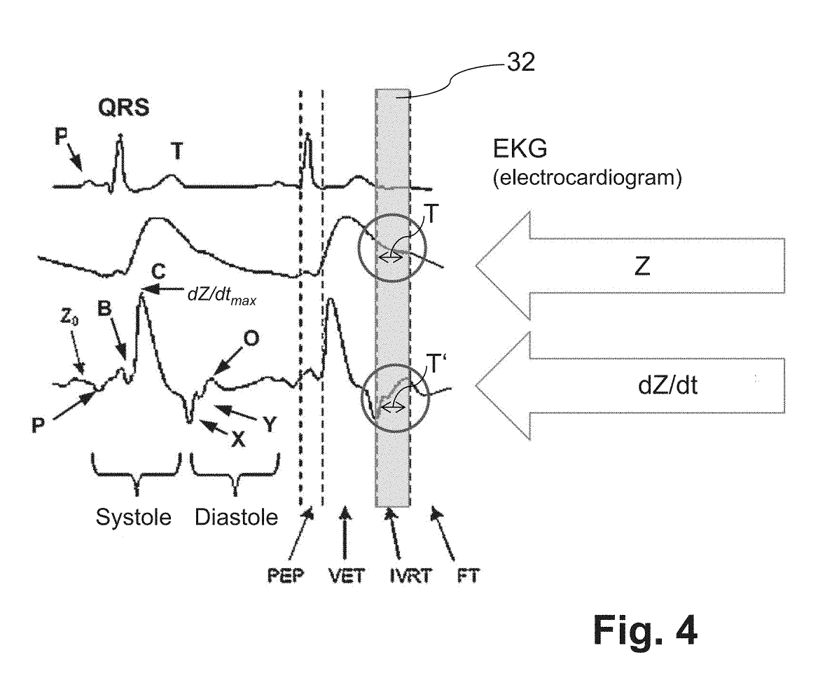

FIG. 3 shows a typical trace of an electrocardiogram of two cardiac cycles, the corresponding impedance Z measurements its first derivative dZ/dt. The highlighted area 31 marks a systolic phase of the heart. For evaluation of the impedance Z or a parameter derived therefrom according to the invention, the signals as shown can for instance be acquired with and without VNS, followed by signal analysis and comparison, for example with such signal parts acquired during diastole, as shown in highlighted area 32 of FIG. 4.

Particularly, the efficacy of VNS can be quantified in relation to how long (time period T) the continuous Impedance waveform Z remains flat during the isovolumetric relaxation period IRVT (see upper arrow in FIG. 4). If lusitropy is improving due to VNS, the amount of time spent in this flat region will shorten. An alternative indicator can be found in the first-order derivative of the intracardiac impedance dZ/dt which will reveal the closure of the aortic valve and the opening of the mitral valve. The length of time between these two changes in valve state is also reduced with effective VNS. One approach to evaluate waveform flatness is to determine amplitude variation of the waveform. For instance, a waveform can be declared as flat when the amplitude has not varied for more than a certain percentage within a predetermined time. An exemplary process for defining flatness of an impedance waveform is given in the following: The impedance signal is sampled at a certain rate. A linear model is generated for the time/impedance pairs (such as z(t)˜m*t+b) for t=0, 1, . . . , n). An analysis of variance is applied and the resulting parameters are tested against the hypothesis that they differ significantly from the null hypothesis of a horizontal line (that is ‘flat’). Since real physiologic signals even when sampling a ‘flat’ region will likely contain some non-zero offset and variance, these statistical moments can be tested with p-value and F-statistic to see if the ‘flat’ region varies significantly from an ideal horizontal line at a fixed direct current value.

Additional methods of establishing VNS efficacy in improving cardiac function include differential estimates of inotropy and lusitropy via measurements of the intracardiac impedance at systole and diastole, respectively. In cases of both heart failure with reduced ejection fraction (HFrEF, systolic HF) and preserved ejection fraction (HFpEF, diastolic HF) the differential measure of dZ/dt at these time points improves with therapy and improved cardiac function.

Particularly, according to the invention, VNS is delivered by the system 1 with a duty cycle ‘on’ period of 10-30 seconds and an ‘off’ period of 30 seconds to 5 minutes. Measurements of the parameter according to the invention are taken during VNS ‘on’ periods and compared against VNS ‘off’ periods, allowing 5-60 seconds for the VNS effect to wash out, provide a rapid efficacy estimate of VNS during normal device operation as well as the initial VNS up-titration period after implant.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A system for evaluating an efficacy of vagus nerve stimulation, the system comprising:
a neurostimulator configured to perform vagus nerve stimulation; and
a measuring component for evaluating the efficacy based on at least one parameter that is indicative of a myocardial contractile state of the heart.

2. The system of claim 1, wherein the parameter is:
intracardiac impedance;
ventricular wall motion;
heart sounds;
low frequency fluid motion acoustic signals; or
a parameter derived therefrom,
wherein the measuring component is configured to measure the parameter.

3. The system according to claim 2, wherein the intracardiac impedance is measured in a unipolar manner, and wherein the measuring component comprises an electrode having a tip that is configured to be arranged at a location in the heart, the location of the heart including an apex of the right ventricle of the heart for measuring the intracardiac impedance.

4. The system according to claim 1, wherein the neurostimulator is configured to activate parasympathetic ganglia in the heart, and wherein for activating the ganglia the neurostimulator is configured to generate electrical impulses and to apply them via at least one or a plurality of stimulation electrodes.

5. The system according to claim 1, wherein the system is configured to determine the parameter via the measuring component during diastole and/or systole of the cardiac cycle.

6. The system according to claim 1, wherein the system is configured to repeatedly determine the parameter during vagus nerve stimulation and in an absence of vagus nerve stimulation and to compare a parameter obtained during vagus nerve stimulation with a parameter obtained in the absence of vagus nerve stimulation for evaluating an efficacy, wherein the comparison is performed by evaluating the parameter with respect to: a reference value, an upper and lower limit, a statistical moment, one or more direct or derived value from the same sensor at another time in the heart cycle, a direct or derived value from another sensor or sensors, or a state of a therapy device.

7. The system according to claim 1, wherein the system further comprises an accelerometer configured to detect movements of the patient.

8. The system according to claim 2, wherein the derived parameter corresponds to:
a time period representing a waveform of the intracardiac impedance or ventricular wall motion, wherein heart sounds or acoustic signals remains flat during the isovolumetric relaxation period; or
a time period between a closure of the aortic valve and an opening of the Mitral valve of the heart, which time period is estimated via a first-order derivative of the measured intracardiac impedance, ventricular wall motion, heart sounds, or acoustic signals waveform.

9. A method for evaluating an efficacy of vagus nerve stimulation, the method comprising:
providing a system according to claim 1; and
evaluating an efficacy of vagus nerve stimulation based on at least one parameter that is indicative of a myocardial contractile state of the heart.

10. The method of claim 9, wherein the parameter includes:
intracardiac impedance;
ventricular wall motion;
heart sounds;
low frequency fluid motion acoustic signals; or
a parameter derived therefrom,
wherein the parameter is measured.

11. The method according to claim 10, wherein the intracardiac impedance is an unipolar intracardiac impedance that is measured using an electrode having a tip that has been arranged at the apex of the right ventricle.

12. The method according to claim 9, wherein the parameter is determined during diastole and/or systole of the cardiac cycle.

13. The method according to claim 9, wherein the parameter is repeatedly determined during vagus nerve stimulation and in an absence of vagus nerve stimulation, and wherein the parameter obtained during vagus nerve stimulation is compared to the parameter obtained in the absence of vagus nerve stimulation for evaluating the efficacy.

14. The method according to claim 9, further comprising:
detecting a movement of the patient; and
deriving an activity measure of the patient from the detected movements.

15. The method according to claim 10, wherein the derived parameter corresponds to:
a time period representing a waveform of an intracardiac impedance, a ventricular wall motion, heart sounds, or acoustic signals that remain flat during the isovolumetric relaxation period; or
a time period between a closure of the aortic valve and an opening of a Mitral valve of the heart, wherein the time period is estimated via a first-order derivative of the measured intracardiac impedance, ventricular wall motion, heart sounds, or acoustic signals waveform.
